(19) United States
(12) Patent Application Publication
(10) Pub. No.: US 2017/0252548 A1
(43) Pub. Date: Sep. 7, 2017

(54) TOOL HUB AND ACCESS POINT AND METHOD

(71) Applicant: COVIDIEN LP, MANSFIELD, MA (US)

(72) Inventor: WILLIAM S. KRIMSKY, FOREST HILL, MD (US)

(21) Appl. No.: 15/450,284

(22) Filed: Mar. 6, 2017

Related U.S. Application Data
(60) Provisional application No. 62/304,391, filed on Mar. 7, 2016.

(51) Int. Cl. A61M 39/02; A61B 18/18; A61B 1/00; A61B 17/34; A61F 2/82; A61B 34/20; A61M 25/10; A61B 90/00; A61B 10/02; A61B 1/267

(52) U.S. Cl. CPC A61M 39/0247; A61B 10/02; A61B 18/1815; A61B 1/00165; A61B 1/2676; A61F 2/82; A61B 34/20; A61M 25/10; A61B 90/39; A61B 17/34; A61B 2018/00577

(57) ABSTRACT

Methods for marking the location of and/or providing for an access point on a tissue wall include navigating an extended working channel (EWC) to an access point and piercing a tissue wall by piercing tool at the access point to create an opening through which the EWC may pass. The opening allows for the deployment of one or more of a diagnostic, imaging, or therapeutic modality from the EWC. Following removal of the one or more diagnostic, imaging, or therapeutic modality, a coagulant may be deposited to permit the opening to close. The location of the piercing is marked to easily identify the location at which the tissue wall was pierced for subsequent procedures.

### CROSS REFERENCE TO RELATED APPLICATION

The present application claims the benefit of and priority to U.S. Provisional Application Ser. No. 62/304,391, filed on Mar. 7, 2016, the entire contents of which are incorporated herein by reference.

### BACKGROUND

Technical Field

The present disclosure relates to a method and system of marking the location of and providing for an access point on a tissue wall. In particular, a visual marker or an access port at the access point facilitates access of medical instruments to target tissue located beyond the tissue wall.

Background Information

A variety of minimally-invasive procedures have been developed with the advance of various medical technologies. Prior to these advances, procedures to obtain tissue biopsies, deliver localized drugs, or place markers, often required large incisions, or open surgery that left large wounds and scars which required extended time to heal. Minimally-invasive technologies now permit many of these procedures to be performed with smaller incisions, reducing both healing time and trauma to a patient. Although minimally-invasive procedures result in lower trauma to a patient, they are often repeatedly performed to access the same location or target tissue in a patient. For example, when taking multiple biopsies over an extended period of time, it is often desirable to obtain each biopsy at the same location in order to properly analyze and assess treatment over time. This repeated access to the same tissue through minimally-invasive procedures results in multiple wounds at different locations where tissue is pierced to allow access for medical instruments. Each time tissue is pierced, it can lead to tissue granulation and a decrease in tissue strength.

In order to minimize the trauma to tissue during re-access to tissue or internal organs, it is desirable to pierce tissue through the same access point as in previous procedures. Thus, there is a need for a method of identifying and marking previous access points within tissue and/or providing for access ports to provide structure to tissue and aid the navigation of medical instruments to target tissue.

### SUMMARY

The present disclosure provides a method for marking a location on a tissue wall, such as an airway wall. The method includes navigating an extended working channel (EWC) to an access point, extending a piercing tool from the EWC, marking the location of the piercing, piercing the tissue wall at the access point to create an opening through which the EWC may pass, and deploying one or more of a diagnostic, imaging, or therapeutic modality from the EWC. The opening created by the piercing tool is then permitted to close following removal of the one or more diagnostic, imaging, or therapeutic modality.

In embodiments, the marking is a coagulant or a permanent dye marker such as a spot marker, tattoo, or fluorescent dye. In other embodiments, the marking is an access port positioned at the access point to permit access through the tissue wall. In embodiments, the access port includes a lattice structure configured to promote tissue growth. The access port may also be coated with a coagulant or a drug configured to promote tissue regrowth. In other embodiments, the access port is bioabsorbable. According to further aspects of the disclosure, the piercing tool is a balloon catheter configured to deploy the access port when inflated. The piercing tool may be configured to deposit a coagulant to prevent bleeding at the access point.

In another embodiment, the access point is identified in a pre-procedure image. The method may further include generating a pathway plan to the access point for navigation of the EWC. The pre-procedure images and the pathway plan may be registered to a location of a patient. In embodiments, navigation of the EWC employs electromagnetic navigation. Following the closure of the opening, the EWC may be re-navigated to the access point in a subsequent procedure.

According to further aspects of the disclosure the diagnostic modality is a biopsy device. The imaging modality may employ a fiber optic lightpath. Additionally, the treatment modality may be a microwave ablation catheter, a chemical ablation applicator, a cryogenic ablation applicator, a radio frequency ablation applicator, a bi-polar resection device, an electrosurgical vessel sealing device, or an ultrasonic vessel sealing device.

### BRIEF DESCRIPTION OF THE DRAWINGS

FIG. 1 is a schematic illustration of an electromagnetic navigation (EMN) system configured for use with an access port in accordance with an illustrative embodiment of the present disclosure;

FIG. 2 is a schematic view of a lung illustrating the use of an access port in accordance with an embodiment of the present disclosure;

FIGS. 3A, 3B, 3C, 3D, and 3E illustrate the navigation and identification of an access point in accordance with an embodiment of the present disclosure; and

FIG. 4 is a schematic view of a scaffold placed within an airway in accordance with an embodiment of the present disclosure.

### DETAILED DESCRIPTION

Described herein are systems and methods for identifying, marking, and/or providing for an access point to tissue and/or organs. In particular, described herein are systems and methods of identifying and navigating to a desired access point on tissue wall and providing for an access port through the tissue wall. Alternatively, another aspect of the current disclosure is to provide for a method of identifying and marking an access point on a tissue wall for easy identification and use during future medical procedures. Even further, another aspect of the current disclosure is to provide a method for identifying an access point and providing for adequate coagulation to reestablish tissue integrity at the access point. Adequate coagulation may be provided by applying a coagulant at the access point.

Detailed embodiments of the present disclosure are disclosed herein. Although the present disclosure describes systems and methods for use in minimally invasive procedures of the airways, the disclosed embodiments are merely examples of one particular medical use and are not intended to be limited to use in patient airways. The disclosed systems and methods may be used in a variety of minimally invasive medical procedures involving various parts of the body, as mentioned below. Therefore, specific structural and functional details disclosed herein are not to be interpreted as limiting, but merely as a representative basis for teaching one skilled in the art to employ the present disclosure in a particular procedure.

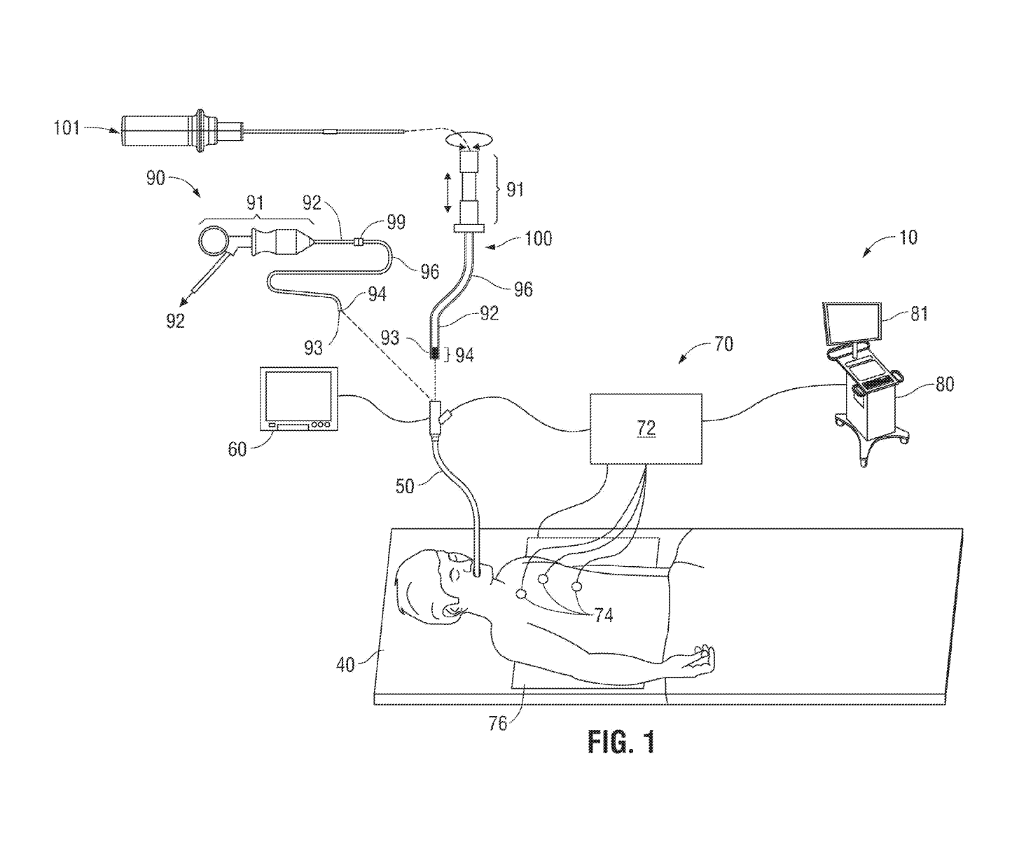

FIG. 1 is an illustration of an electromagnetic navigation (EMN) system 10 in accordance with the present disclosure and is an exemplary embodiment of a method of navigating to a desired point in a patient airway. One such EMN system is the ELECTROMAGNETIC NAVIGATION BRONCHOSCOPY® system currently sold by Medtronic, Inc. The EMN system 10 may be used for planning and generating a pathway to target tissue or an access point and navigating a biopsy tool to the target tissue to obtain a tissue sample from the target tissue. A series of pre-procedure images of the patient airways are obtained using one or more imaging modalities, including the use of computerized tomography (CT) scans, and used for planning and generating the pathway to the target. During an EMN procedure, the patient's location is registered to the pre-procedure images, or generated pathway.

EMN system 10 generally includes an operating table 40 configured to support a patient, a bronchoscope 50 configured for insertion through the patient's mouth and/or nose into the patient's airways, monitoring equipment 60 coupled to bronchoscope 50 for displaying video images received from bronchoscope 50, a tracking system 70 including a tracking module 72, a plurality of reference sensors 74, an electromagnetic field generator 76, and a workstation 80 including software and/or hardware used to facilitate pathway planning, identification of target tissue, and navigation to target tissue.

FIG. 1 also depicts two types of catheter guide assemblies 90, 100. Both catheter guide assemblies 90, 100 are usable with the EMN system 10 and share a number of common components. Each catheter guide assembly 90, 100 includes a handle 91, which is connected to an extended working channel (EWC) 96. The EWC 96 is sized for placement into the working channel of bronchoscope 50. In operation, a locatable guide (LG) 92, including an electromagnetic (EM) sensor 94, is inserted into the EWC 96 and locked into position such that the sensor 94 extends a desired distance beyond the distal tip of the EWC 96. In one embodiment, the LG 92 is integrated with the EWC 96 so the EM sensor 94 is disposed on the EWC 96. The location of the EM sensor 94, and thus the distal end of the EWC 96, within an electromagnetic field generated by the electromagnetic field generator 76 can be derived by the tracking module 72, and the workstation 80. Catheter guide assemblies 90, 100 have different operating mechanisms, but each contain a handle 91 that can be manipulated by rotation and compression to steer a distal tip 93 of the LG 92 and extended working channel (EWC) 96. Catheter guide assemblies 90 are currently marketed and sold by Medtronic, Inc. under the name SUPERDIMENSION® Procedure Kits. Similarly catheter guide assemblies 100 are currently sold by Medtronic, Inc. under the name EDGE™ Procedure Kits. Both kits include a handle 91, extended working channel 96, and locatable guide 92. For a more detailed description of the catheter guide assemblies 90, 100 reference is made to commonly-owned U.S. Pat. No. 9,247,992 filed on Mar. 15, 2013 by Ladtkow et al., the entire contents of which are hereby incorporated by reference.

As illustrated in FIG. 1, the patient is shown lying on an operating table 40 with a bronchoscope 50 inserted through the patient's mouth and into the patient's airways. Bronchoscope 50 may include a source of illumination and a video imaging system (not explicitly shown) and is coupled to monitoring equipment 60, e.g., a video display, for displaying the video images received from the video imaging system of bronchoscope 50.

Catheter guide assemblies 90, 100 including LG 92 and EWC 96 are configured for insertion through a working channel of bronchoscope 50 into the patient's airways (although the catheter guide assemblies 90, 100 may alternatively be used without bronchoscope 50). In catheter guide assembly 90, the LG 92 and EWC 96 are selectively lockable relative to one another via a locking mechanism 99. Alternatively, an EM sensor 94 may be disposed directly on the EWC 96, as described above. A six degrees-of-freedom electromagnetic tracking system 70, e.g., similar to those disclosed in U.S. Pat. No. 6,188,355 and published PCT Application Nos. WO 00/10456 and WO 01/67035, the entire contents of each of which are incorporated herein by reference, or any other suitable positioning measuring system is utilized for performing navigation, although other configurations are also contemplated. Tracking system 70 is configured for use with catheter guide assemblies 90, 100 to track the position of the EM sensor 94 as it moves in conjunction with the EWC 96 through the airways of the patient, as detailed below.

As shown in FIG. 1, electromagnetic field generator 76 is positioned beneath the patient. Electromagnetic field generator 76 and the plurality of reference sensors 74 are interconnected with tracking module 72, which derives the location of each reference sensor 74 in six degrees of freedom. One or more of reference sensors 74 are attached to the chest of the patient. The six degrees of freedom coordinates of reference sensors 74 are sent to workstation 80, which includes application 81 where sensors 74 are used to calculate a patient coordinate frame of reference.

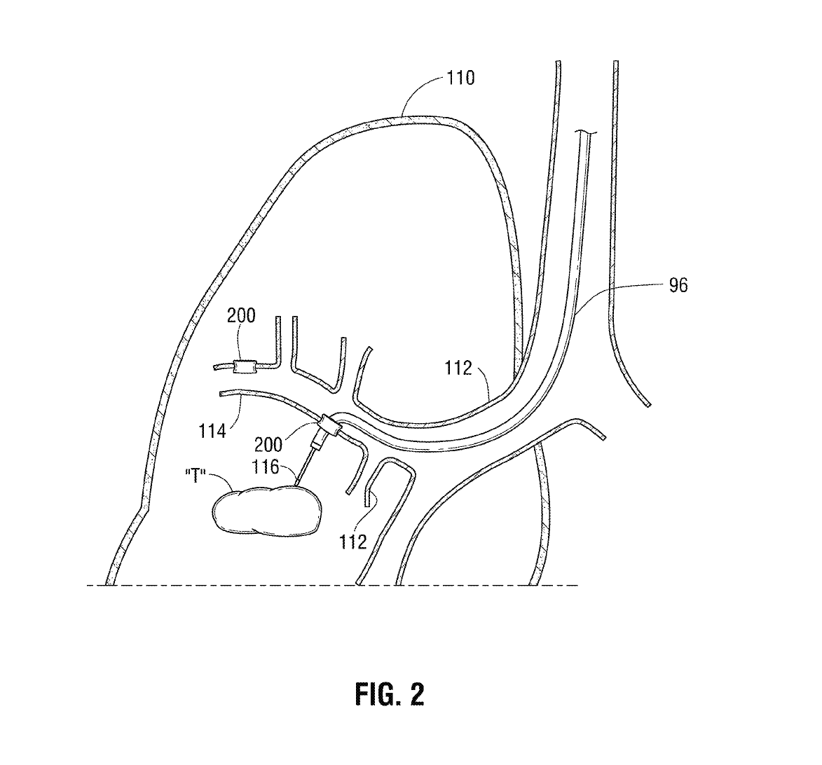

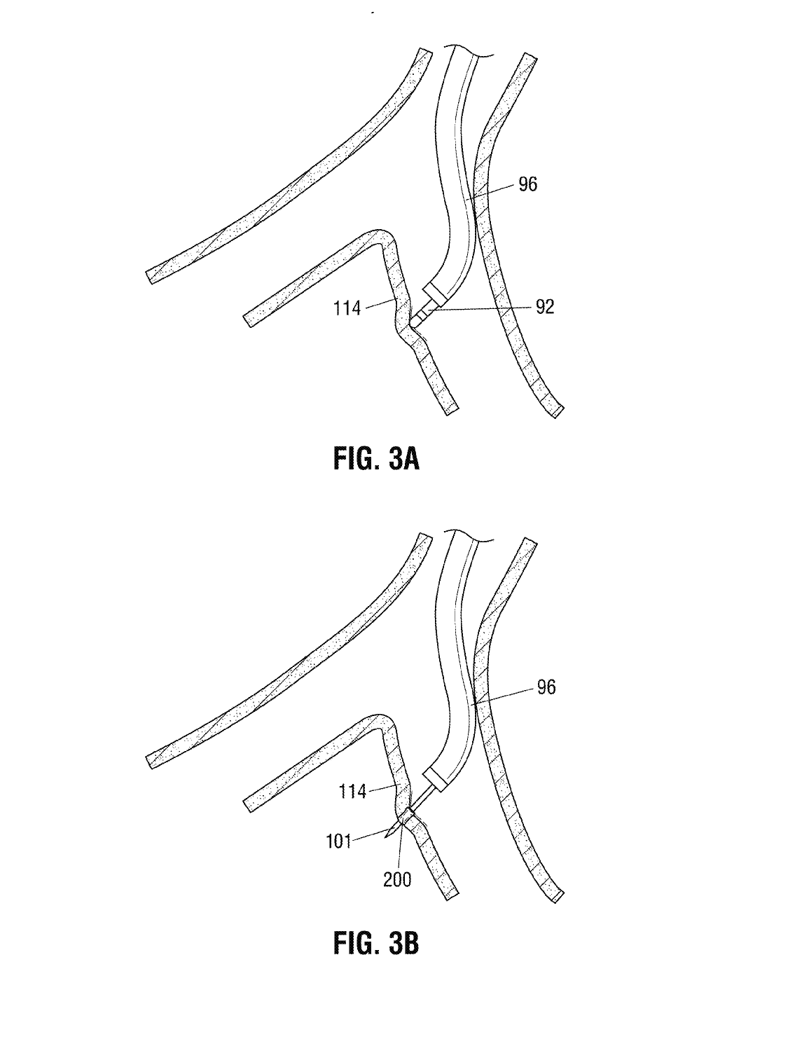

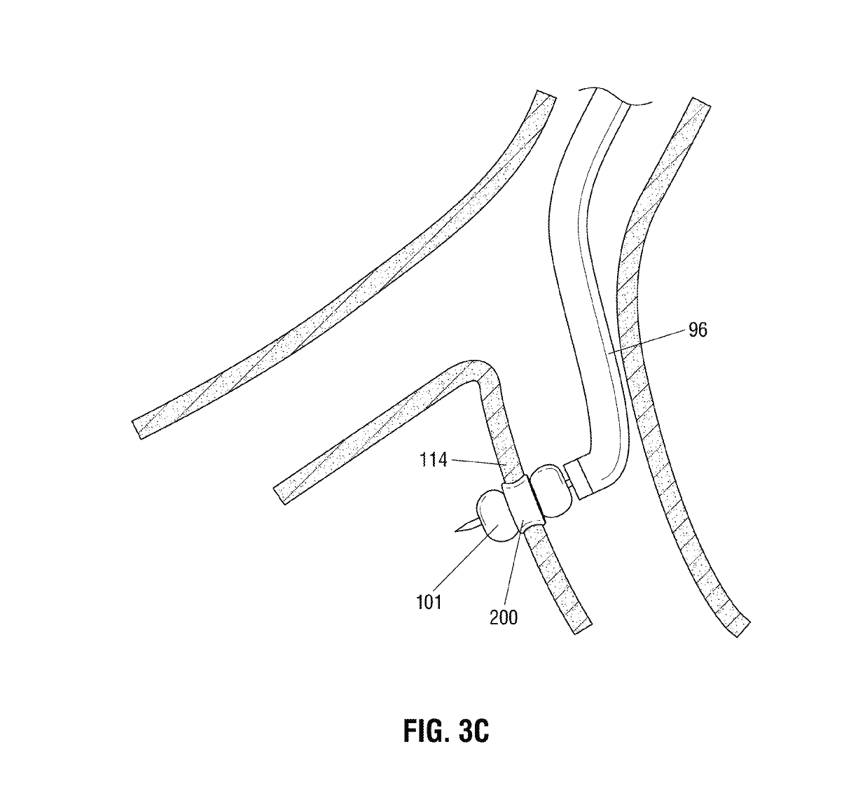

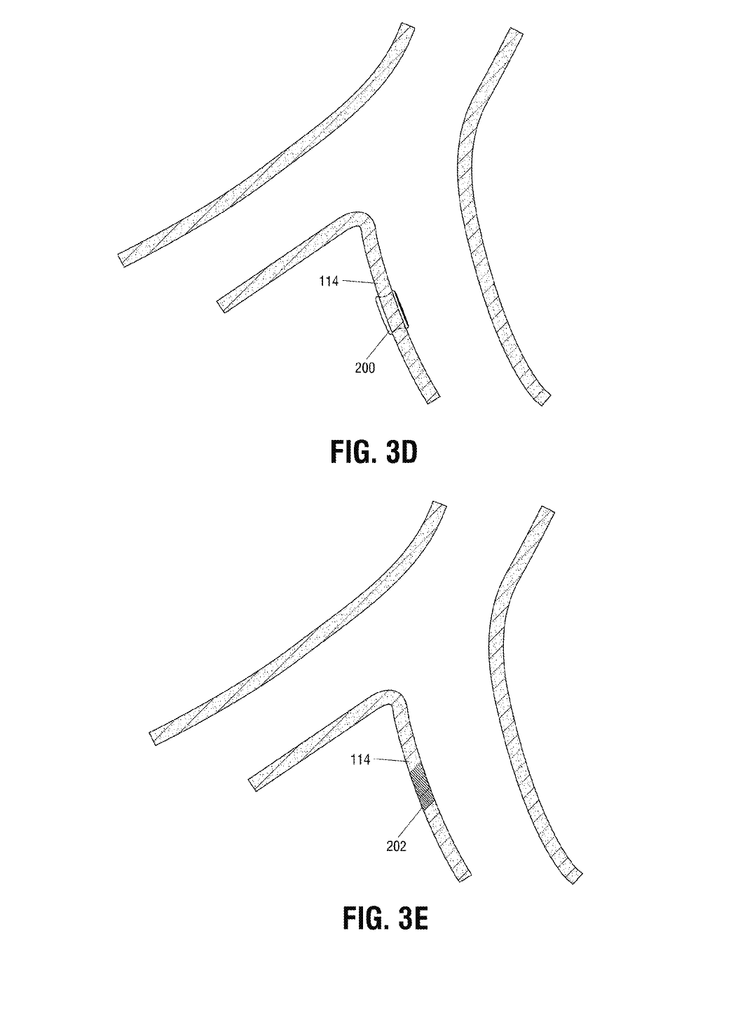

In practice, a clinician uses the catheter guide assemblies 90, 100 to navigate the EWC 96 using the EM sensor 94 to reach a desired access point from within the luminal network of the lungs (e.g. the airways). Once the desired access point is reached, a placement catheter 101 is inserted into the EWC 96. The placement catheter 101 (shown in connection with catheter guide assembly 100) is then extended from the EWC 96 to pierce the bronchial walls and place an access port 200 through the airway wall, as shown in FIG. 2. In embodiments, the placement catheter 101 (shown in FIGS. 3B and 3C) is configured to pierce the bronchial airway walls. In other embodiments, a separate bronchial piercing catheter is used to pierce the airway walls prior to inserting the placement catheter 101. Once the access port 200 is in place, the placement catheter 101 is removed, and a biopsy tool or other medical tool such as an ablation catheter is inserted into the EWC 96 and advanced through the access port 200 to the target tissue. It can also serve as a means to reestablish tissue integrity as well as for appropriate coagulation at that site independent of whether an access port in placed or not. The placement of the access port 200 can also be used as a means to identify and locate the same access point for future procedures.

FIG. 2 depicts an access port 200 placed within the bronchial airways 112 of a patient lung 110. In particular, access port 200 is placed through airway walls 114. In this embodiment, a catheter or medical instrument 116 can be placed through an EWC 96 which is navigated through the bronchial airways 112 and can access target tissue “T,” located outside the airway walls 114, through access port 200.

FIGS. 3A-3D illustrate one embodiment of the placement of access port 200 through an airway wall 114 by a technician. FIG. 3A depicts the navigation of an EWC 96 to a desired access point in the airway wall 114. Once the EWC 96 reaches the desired access point, the technician removes the LG 92 from the EWC 96 and replaces it with a piercing catheter or a placement catheter 101. As described above, the EM sensor 94 may be directly integrated with the EWC 96, thereby eliminating the need for an LG 92. The placement catheter 101 pierces the airway wall 114 with a needle located on the distal end of the catheter 101, as depicted in FIG. 3B. In the embodiment depicted in FIG. 3B, the placement catheter 101 is configured to both pierce the airway wall and carry an access port 200 in an un-deployed state. In an alternative embodiment (not shown), the piercing catheter and the placement catheter are separate catheters. In FIG. 3C, the placement catheter 101 deploys the access port 200. In this embodiment, the placement catheter 101 includes a balloon catheter configured to expand to deploy and place the access port 200 through the access point. FIG. 3D depicts the access port 200 fully deployed and placed through the airway wall 114. The access port 200 provides a passage for various medical instruments, or diagnostic, imaging, treatment, or therapeutic modalities, to exit the airway wall to access target tissue “T.” For example, the access port 200 may provide a passage for a biopsy device, a fiber optic lightpath for imaging, or a microwave ablation catheter. The access port 200 may also provide a passage for treatment modalities such as a microwave ablation catheter, a chemical ablation applicator, a cryogenic ablation applicator, a radio frequency ablation applicator, a bi-polar resection device, an electrosurgical vessel sealing device, or an ultrasonic vessel sealing device. In embodiments, the access port 200 is configured to promote tissue regrowth through the access port 200, as depicted in FIG. 3D. For example, access port 200 may comprise a lattice structure to encourage tissue regrowth and allow the access port 200 to be low weight. In another embodiment, access port 200 is bioabsorbable.

Additionally, the access port 200 may be coated with a coagulant or treated with a composition to promote tissue regrowth. The access port 200 provides for structural support of the airway wall 114 when tissue regrows and seals the pierced airway wall 114. During subsequent procedures, a technician can use the access port 200 as a visual marker and a means to identify and locate the previous access point. Rather than create a new piercing of the airway wall 114, the technician can simply re-pierce the airway wall 114 at the same location. This limits the areas of the airway wall 114 subject to tissue granulation and degradation. In an embodiment, the access port includes sensors that include location based sensors, for example, electromagnetic sensors, that can be detected externally. These sensors allow for the measuring of changes in position either relative to itself or to another point. Additionally, these sensors could be configured to assess the local environment, for example, chemical sensors, temperature sensors, pH sensors, etc.

In an embodiment, the access port 200 can include an internal port or reservoir. In particular, the internal port or reservoir may store therapeutic drugs for delivery to the area in which it is placed or to areas distal to the access port. In this manner, the internal port or reservoir can be accessed, refilled, interrogated, etc, via the bronchoscope or other minimally invasive technique depending on what organ system or tissue is being accessed.

In another embodiment, after the placement catheter 101, or piercing catheter, pierces the tissue wall, the placement catheter is configured to deposit a coagulant 202 or similar drug to help prevent bleeding and/or promote tissue regrowth at the access point, as depicted in FIG. 3E. The coagulant helps to reestablish tissue integrity at the access point. This embodiment can be an alternative to the placement of an access port 200 at the access point or use in combination therewith.

In an alternative embodiment, the placement catheter 101 also functions as a marking catheter (not shown). Alternatively, the marking catheter may be a separate catheter used in place of, or in combination with the placement catheter 101. The marking catheter places an identifying mark at an access point, either before or after the airway wall 114 is pierced. The identifying mark can be a permanent dye marker such as a simple spot marker, tattoo, fluorescent dye, or other type of compound to identify the access point such that during a subsequent follow up procedure, that area can be identified. Additionally, the identifying mark can be used to track the access point's location with respect to a target tissue or used to assess local biome environment.

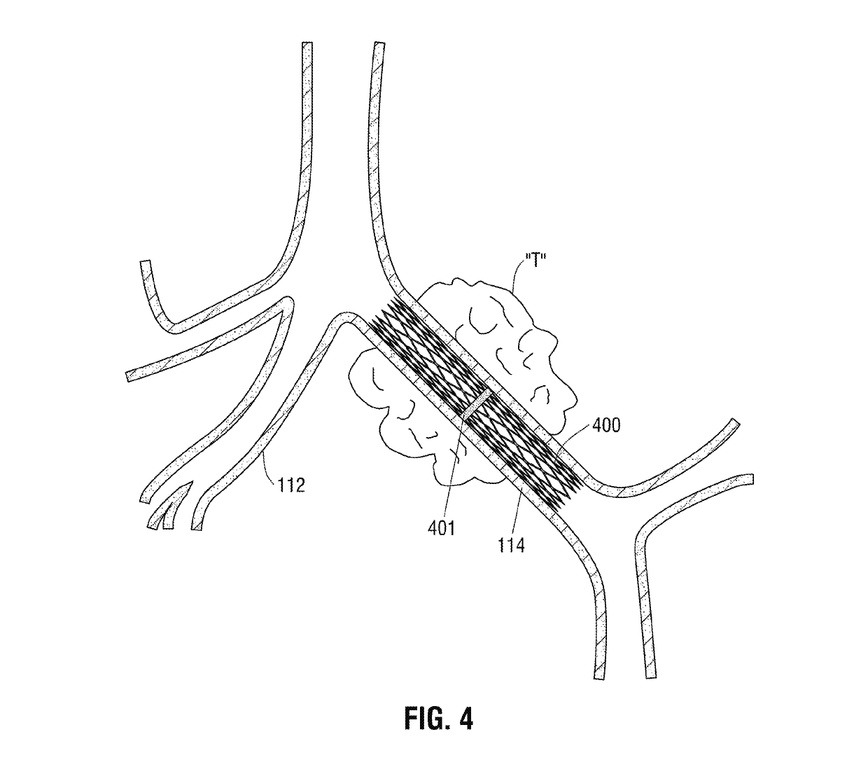

FIG. 4 depicts an alternative embodiment in which a scaffold 400 is placed parallel to the airway walls 114. In this embodiment, scaffold 400 has a mesh or lattice configuration to encourage tissue regrowth and may provide structural integrity to the airway wall 114. Since multiple incisions or punctures to the airway wall 114 can cause tissue granulation and structural degradation, scaffold 400 helps to provide structural integrity and support to the airway walls 114. Additionally, the scaffold 400 includes at least one visual marker 401 which identifies a desired access point or area where the tissue wall was previously pierced to allow for a catheter to pierce the airway walls 114. This allows a technician to re-access target tissue “T” through the same access point to prevent degradation and tissue granulation of airway walls 114 at multiple points. The visual marker may be a colored band 401, a change in texture or appearance of the scaffold, or any other type of visual reference point placed on the scaffold 400 or embedded in the material that comprises the scaffold 400. The scaffold 400 may also include a radioactive marker, a navigational beacon, an acoustic sensor, or a chemical sensor to help identify the location of the scaffold 400 in the airways and may include other visual indicators. In this embodiment, a number of different diagnostic or therapeutic devices may also be incorporated into the scaffold 400. The scaffold 400 may include a reservoir configured to store and release therapeutic agents, wherein the scaffold 400 would allow the technician to identify the area and refill the reservoir during subsequent procedures.

The access port 200 or scaffold 400 can also be used as a point of reference when evaluating treatment of target tissue “T.” For example, the location of target tissue “T” and its movement over time relevant to the spot marker, access port 200, or scaffold 400 on the airway wall can be assessed. The change in location of the target tissue “T” relevant to a fixed location in the body may provide probative value in the treatment of the patient. For example, the change in location of target tissue “T” in one direction, relative to the spot marker, access port 200, or scaffold 400, may indicate successful treatment, while movement in a different direction may indicate unsuccessful treatment.

Detailed embodiments of devices, systems incorporating such devices, and methods using the same have been described herein. However, these detailed embodiments are merely examples of the disclosure, which may be embodied in various forms. Therefore, specific structural and functional details disclosed herein are not to be interpreted as limiting, but merely as a basis for the claims and as a representative basis for allowing one skilled in the art to employ the present disclosure in virtually any appropriately detailed structure. While the preceding embodiments were described in terms of bronchoscopy of a patient's airways, those skilled in the art will realize that the same or similar devices, systems, and methods may be used in other lumen networks, such as, for example, the vascular, lymphatic, genitourinary and/or gastrointestinal networks as well or other solid organ systems such as the liver, kidneys, pancreas, etc.

What is claimed is:

1. A method for marking a location on tissue wall, the method comprising:
navigating an extended working channel (EWC) to an access point in a tissue wall;
extending a piercing tool from the EWC;
piercing the tissue wall at the access point to create an opening through which the EWC may pass;
deploying one or more of a diagnostic, imaging, or therapeutic modality from the EWC;
marking the location of the piercing; and
permitting the opening to close following removal of the one or more diagnostic, imaging, or therapeutic modality.

2. The method of claim 1, wherein the marking is a coagulant or a permanent dye marker.

3. The method of claim 2, wherein the permanent dye marker is a spot marker, tattoo, or fluorescent dye.

4. The method of claim 1, wherein the marking is an access port positioned at the access point to permit access through the tissue wall.

5. The method according to claim 4, wherein the piercing tool is a balloon catheter configured to deploy the access port when inflated.

6. The method according to claim 4, wherein the access port comprises a lattice structure configured to promote tissue growth.

7. The method according to claim 4, wherein the access port is coated with at least one of a coagulant and a drug configured to promote tissue regrowth.

8. The method according to claim 1, wherein the tissue wall is an airway wall.

9. The method according to claim 4, wherein the access port is bioabsorbable.

10. The method according to claim 4, wherein the piercing tool is configured to deposit a coagulant to prevent bleeding at the access point.

11. The method according to claim 1 further comprising identifying the access point in a pre-procedure image.

12. The method according to claim 11 further comprising generating a pathway plan to the access point for navigation of the EWC.

13. The method according to claim 12 comprising registering the pre-procedure images and the pathway plan to a location of a patient.

14. The method according to claim 13 wherein navigation of the EWC employs electromagnetic navigation.

15. The method according to claim 1 further comprising re-navigating the EWC to the access point following closure of the opening.

16. The method according to claim 15 wherein the re-navigation occurs in a subsequent procedure.

17. The method according to claim 1, wherein the diagnostic modality is a biopsy device.

18. The method according to claim 1, wherein the imaging modality employs a fiber optic lightpath.

19. The method according to claim 1, wherein the treatment modality is a microwave ablation catheter.

20. The method according to claim 1, wherein the treatment modality is selected from the group consisting of a chemical ablation applicator, a cryogenic ablation applicator, a radio frequency ablation applicator, a bi-polar resection device, an electrosurgical vessel sealing device, and an ultrasonic vessel sealing device.
